(19) United States
(12) Patent Application Publication
(10) Pub. No.: US 2008/0206876 A1
(43) Pub. Date: Aug. 28, 2008

(54) INSTRUMENTS FOR THE SELECTIVE AND DIRECT DETECTION OF FREE METALS IN FLUIDS AND METHODS TO DIAGNOSE METAL-RELATED DISEASES AND DETERMINE PHARMACOLOGIC DOSING REGIMENS

(76) Inventors: Steve H. Kanzer, Ann Arbor, MI (US); John S. Althaus, Ann Arbor, MI (US)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(21) Appl. No.: 11/871,935

(22) Filed: Oct. 12, 2007

Related U.S. Application Data
(60) Provisional application No. 60/829,252, filed on Oct. 12, 2006.

(51) Int. Cl. G01N 33/20; G01N 27/26; G01N 33/00

(52) U.S. Cl. 436/34; 204/412; 205/775; 436/74

(57) ABSTRACT

Instruments for the selective and direct detection of free metals in fluids and methods to diagnose metal-related diseases and determine pharmacologic dosing regimens are disclosed.

### CROSS REFERENCE TO RELATED APPLICATIONS

This application claims priority from U.S. Provisional Application Ser. No. 60/829,252, filed Oct. 12, 2006, the contents of which are incorporated herein by reference.

### BACKGROUND

A number of diseases and health conditions have been linked to lower or higher serum metal levels, such as copper and iron. Convenient direct measurement of free and bound copper and iron in body fluids is a significant medical need. Measurement of copper and iron provides functions that are diagnostic, prognosticative and/or maintenance-serving. Prior to the present invention, currently available methods of estimating free and bound copper and iron in plasma and serum rely upon estimations that are subject to a considerable amount of variability and inaccuracy. For example, the current “gold standard” for measuring “free serum copper” involves a measurement of total serum copper (generally determined by flame absorbance spectroscopy) and subtracting the estimated amount of copper theoretically bound to the serum protein ceruloplasmin. Such estimation, however, can be highly inaccurate, however, due to the variation in actual copper-ceruloplasmin binding that varies between individuals as well as factors such as aging during a person's lifetime. For example, it is widely assumed that a single ceruloplasmin protein binds seven copper atoms. However, with aging the binding capacity of ceruloplasmin can be as low as five copper atoms. Since an elevated pool of free copper in serum can be toxic to the central nervous system and other organs, what is needed, and the present invention provides, is an instrument and methodology capable of directly measuring free and bound copper without relying on estimations that may or may not be correct. A preferred embodiment of the present invention also provided a method of diagnosing persons having potentially toxic elevated free copper pools as well as means of dosing one or more copper lowering agents based upon direct measurements of free serum copper pools. Examples of diseases that may be associated with elevated free serum copper pools include, Wilson's disease, Alzheimer's disease, Parkinson's disease, schizophrenia, atherosclerosis, diabetes, and other common diseases.

### Copper Related Diseases:

### Wilson's Disease:

Wilson's disease is a rare autosomal recessive inherited disorder of copper metabolism. The condition is characterized by excessive deposition of copper in the liver, brain, and other tissues. The major physiologic aberration is decreased excretion of copper by the liver. The genetic defect, localized to chromosome arm 13q, has been shown to affect the copper-transporting adenosine triphosphatase (ATPase) gene (ATP7B) in the liver. Patients with Wilson's disease usually present with liver disease during the first decade of life subsequent to neuropsychiatric illness during the second and third decade. The diagnosis is confirmed by measurement of serum ceruloplasmin, urinary copper excretion, serum free copper and hepatic copper content, as well as the detection of Kayser-Fleischer rings.

### Menkes Disease:

Menkes Disease is caused by a defective gene that regulates the metabolism of copper in the body. Because it is an X-linked gene, the disease primarily affects male infants. Copper accumulates at abnormally low levels in the liver and brain, but at higher than normal levels in the kidney and intestinal lining. Affected infants may be born prematurely. Symptoms appear during infancy. Normal or slightly slowed development may proceed for 2 to 3 months, and then there will be severe developmental delay and a loss of early developmental skills. Menkes Disease is also characterized by seizures, failure to thrive, subnormal body temperature, and strikingly peculiar hair, which is kinky, colorless or steel-colored, and easily broken. There can be extensive neurodegeneration in the gray matter of the brain. Arteries in the brain can also be twisted with frayed and split inner walls. This can lead to rupture or blockage of the arteries. Weakened bones (osteoporosis) may result in fractures.

### Alzheimer's Disease:

The relation between serum free copper and Alzheimer's Disease has received considerable attention over the last few years. Copper is an essential element and under normal physiologic circumstances is maintained complexed to proteins. This appears to be a protective mechanism developed in mammals to prevent “free copper” availability. In the free form copper is a highly reactive metal causing free radical formation and oxidation. Normally, most copper is bound to Cp in the serum. In the brain, protective mechanism have also evolved to keep copper complexed to intracellular proteins and those in the CSF. Copper has been shown to be bound to the Tau protein, amyloid precursor protein, the beta secreatase, beta amyloid protein, and apoE. We believe copper is normally bound to these protein as a protective mechanism against excess copper. Indeed, in vitro studies have shown that amyloid precursor protein expression is down regulated in copper depleted cells. The presence of beta amyloid plaques and intracellular neurofibullary tangles appear to result from copper binding to beta-amyloid and the tau protein, respectively, and induction of protein structural changes. This appears to be a pathological mechanism to deal with excess free copper. Thus, under conditions of excess free copper in the brain, these protein structural changes appear to be the markers of the disease and not the cause. It should be noted that in the elderly, two conditions lead to elevated copper in the brain. First, the blood brain barrier become more permeable as we age and thereby allows exchange between the serum and the CSF to occur more easily. Secondly, as liver function diminishes, the amount of copper associated with Cp is decreases, from about 7 copper atoms/Cp molecule to about 5 copper atoms/Cp molecule making free copper more available for transport to the brain.

Interestingly, the work of Squitti and associates have shown that the level of copper unassociated with ceruloplasmin is markedly elevated in AD subjects compared to age-matched controls.

In addition, the work of Sparks et al. Proc Natl Acad Sci USA. 2003 Sep. 16; 100(19):11065-9. Epub 2003 Aug. 14. have shown a direct link between copper in the drinking water in an experimental rabbit model of cholesterol-diet induced Alzheimer's disease. In this work these investigators demonstrated that animals consuming distilled water had markedly reduced AD plaques in the brain compared to tap water controls. Furthermore, they determined that copper was the culprit mineral in the water that induced this effect. The cholesterol-diets likely caused endothelial damage to the blood brain barrier allowing the easy penetration to the brain compartment. The tap water rabbit also suffered dramatically poorer memories in complex tests.

### ALS:

Goto J J, Zhu H, Sanchez R J, Nersissian A, Gralla E B, Valentine I S, Cabelli D E. Loss of in vitro metal ion binding specificity in mutant copper-zinc superoxide dismutases associated with familial amyotrophic lateral sclerosis. J Biol Chem. 2000 Jan. 14; 275(2):1007-14.

The presence of the copper ion at the active site of human wild type copper-zinc superoxide dismutase (CuZnSOD) is essential to its ability to catalyze the disproportionation of superoxide into dioxygen and hydrogen peroxide. Wild type CuZnSOD and several of the mutants associated with familial amyotrophic lateral sclerosis (FALS) (Ala(4) -->Val, Gly(93)-->Ala, and Leu(38)-->Val) were expressed in Saccharomyces cerevisiae. Purified metal-free (apoproteins) and various remetallated derivatives were analyzed by metal titrations monitored by UV-visible spectroscopy, histidine modification studies using diethylpyrocarbonate, and enzymatic activity measurements using pulse radiolysis. From these studies it was concluded that the FALS mutant CuZnSOD apoproteins, in direct contrast to the human wild type apoprotein, have lost their ability to partition and bind copper and zinc ions in their proper locations in vitro. Similar studies of the wild type and FALS mutant CuZnSOD holoenzymes in the “as isolated” metallation state showed abnormally low copper-to-zinc ratios, although all of the copper acquired was located at the native copper binding sites. Thus, the copper ions are properly directed to their native binding sites in vivo, presumably as a result of the action of the yeast copper chaperone Lys7p (yeast CCS). The loss of metal ion binding specificity of FALS mutant CuZnSODs in vitro may be related to their role in ALS.

### Parkinson's Disease:

Forsleff L, Schauss A G, Bier I D, et al. Evidence of functional zinc deficiency in Parkinson's disease. J Altern Complement Med 1999; 5:57-64.

Uitti R J, Rajput A H, Rozdilsky B, et al. Regional metal concentrations in Parkinson's disease, other chronic neurological diseases, and control brains. Can J Neurol Sci 1989; 16:310-4.

Pall H S, Williams A C, Blake D R, et al. Raised cerebrospinal fluid copper concentration in Parkinson's disease. Lancet 1987; 2(8553):238-41.
Dexter D T, Carayon A, Javoy-Agid F, et al. Alterations in the levels of iron, ferritin and other trace metals in Parkinson's disease and other neurodegenerative diseases affecting the basal ganglia. Brain 1991; 114:1953-75.

Copper levels were significantly higher in the cerebrospinal fluid of patients with idiopathic Parkinson's disease than in the control group (Pall et al. 1987). Although the specific reason for elevated copper levels was not known, copper is generally high when there is chronic inflammation, such as that caused by autoimmune or undetected allergy reactions. Furthermore, a copper enzyme is required to convert tyrosine into levodopa. Therefore, elevated levels of brain copper may be an attempt to stimulate production of levodopa. However, high copper levels in the presence of antioxidant deficiencies tend to cause increased free-radical damage to nerve cell DNA.

People with Parkinson's disease have shown both decreased and increased levels of zinc and copper. Both nutrients function in the antioxidant enzyme superoxide dismutase (SOD). SOD tends to be low in the area of the brain involved in Parkinson's disease. In theory, therefore, low levels of zinc and copper could leave the brain susceptible to free radical damage. However, copper and zinc (as well as iron) taken in excess can also act as pro-oxidants, and all have been associated with an increased risk of developing Parkinson's disease in preliminary research. Insufficient evidence currently exists for either recommending or avoiding supplementation with zinc and copper.

### Other Diseases:

Gray hair and skin wrinkles are signs of copper deficiency. Other diseases involving copper deficiency include: Anemia, baldness, benign prostatic hyperplasia, bone and joint abnormalities, brain disturbances, diarrhea, elevated LDL cholesterol levels, general weakness, hypoglycemia, impaired immune function, impaired respiratory function, osteoporosis, retinal degeneration, rheumatoid arthritis and skin sores.

### Detection of Copper Abnormalities:

Imaging technologies including CT scan, PET scan, MRI, ultrasound and nuclear medicine of various organs have been used to detect disease in patients exhibiting copper abnormalities.

The diagnosis of copper abnormalities is typically made by a blood test for serum ceruloplasmin, a liver biopsy for measurement of liver copper content and a test for urinary copper-excretion levels.

Analytical methods for measuring copper in biological and environmental samples include atomic absorption spectrometry, anodic stripping voltammetry, graphite furnace atomic absorption, inductively coupled plasma-atomic emission spectroscopy and inductively coupled plasma-mass spectrometry.

Detection of copper in biological samples for purposes of disease diagnosis is complicated by the fact that copper exits in a variety of discrete and separate pools. In plasma, copper is bound to ceruloplasmin (Log K=˜12), albumin (Log K=˜7), transcuprein (Log K=<7), amino acids (Log K=7 to 5) and to a lesser extent other ligands. In addition, a very small percentage of copper exits free in solution.

Diagnosis of copper-dependent pathologies depends on the distribution of copper within these pools. As such copper can be described as good and bad. Good copper is tightly bound to structures in which the Log K is 12 or greater. Bad copper is loosely bound to structures in which the Log K is 7 or less. Copper is bad when it is loosely bound and available to participate in free radical reactions.

Total copper in biological samples is typically detected using atomic absorption spectrometry in which discernment between pools of copper is not possible. Alternatively, methods which resolve serum into protein and non-protein fractions have been employed. Unfortunately these methods do not discriminate between copper bound to ceruloplasmin, albumin and transcuprein. In this case, protein-bound copper still contain both good and bad copper and the usefulness of this measurement falls into question.

### SUMMARY

The present invention is capable of detecting and measuring non-covalently bound metal (free metal) in bodily fluids that contain both free and bound metal. Discrimination between free and bound metal is based on the kinetics of metal deposition on the surface of an electrode. Once deposited, metal detection and measurement is based on a signal generated using square wave voltammetry. Parameters for both deposition potential and the deposition time can be finely tuned so that detection of free metal only can be performed in a solution from a mixture that contains both free and bound metal. The invention has particular utility in connection with the detection and measurement of non-covalently bound copper (free copper) concentrations in bodily fluids and will be described in detail in connection with such utility although other utilities are contemplated, including detection and measurement of other non-covalently bound metals including but not limited to iron, zinc, manganese, cadmium, selenium or other metal cations.

The invention is suited to a number of diseases in which measurement of free copper in serum can be diagnostic. Most notable is Wilson's disease. Wilson's is a genetic disease in which pathologic copper accumulation affects the health and function of the liver. In Wilson's, total serum copper can be normal or slightly decreased from normal. Total serum copper falls into pools that are bound or free. Most of the bound copper is associated with ceruloplasmin (Cp) which in normal subjects represents about 80% of total copper. The free copper pool is distributed between albumin, peptides and amino acids. In Wilson's, ceruloplasmin can be decreased but it is the free copper that is increased and believed to play a diagnostic as well as a pathologic role.

Our invention for measuring free copper is comparable in ease of use to that used in a glucometer that incorporates electrochemical detection technology. We hypothesis that detection of copper in serum can be discerned because the two pools of copper, free and bound, will have different rates of voltage-mediated deposition onto an electrode.

Our experimental data supports this hypothesis. We found that deposition of copper onto the electrode from copper sulfate (free copper by definition) peaked at 3 min. However, deposition of copper in serum peaked at 30 min. The rate of deposition within the first 3 min was identical for copper as free copper sulfate or in serum. As such, we believe that this 3 min measurement represents free copper. In addition, the amount of copper detected in serum at 3 min was only about 13% of the total copper detected at 30 min. This value is consistent with that based on other published reports.

We also measured free copper in serum samples from Wilson's patients. Free copper was determined in two ways. One way was based on a calculation measuring total copper by atomic absorption and subtracting the bound copper based on a measurement of ceruloplasmin in each sample (X-axis). The other way (Y-axis) was based on the measurement of free copper using a potentiostat by the method described under Experimental. The correlation coefficient describing the linear relationship between both sets of data is r=0.96.

A feature and advantage of the present invention is that it is not dependent upon use of a potentiostat to measure free levels of copper and other metals in bodily fluids. Other techniques to “directly” detect free and bound metal levels which might be applied include, for example, ultrafiltration, immunometric, separation column (i.e. Sephrose), magnetic bead, immobilized metal affinity chromatography, 2D gel electrophersis (i.e. SDS-PAGE) and other protein separation techniques available to those skilled in the art. In these techniques, metal binding proteins of interest, such as ceruloplasmin and transferring may be separated and provided the methodologies are not too denaturing, the level of metals of interest bound to such metalloproteins of interest can be subsequently determined by flame absorbance spectroscopy, for example. This bound metal measurement can then subtracted from the level of total metal of interest in the original fluid sample to arrive at an estimate of the free metal pool of interest in such sample. Such techniques, are however, labor intensive, time consuming, subject to inaccuracies due to metal stripping effects such processes may have on the samples and still rely upon a subtraction estimate to arrive at an estimate of the free metal levels of interest in the fluid sample (such as non-ceruloplasmin bound copper and nontransferrin bound iron (NTBI), as opposed to directly measuring the actual and kinetic availability of the metal of interest in the original sample.

Elevated free metal levels in serum samples can be interest in both the diagnosis and proper treatment of a number of diseases, such as Wilson's disease, iron overload related to blood transfusions, neurodegenerative and CNS diseases, such as, Alzheimer's disease, Parkinson's disease, ALS, Pick's disease, prion-related disease, schizophrenia, diabetes, heart disease and atherosclerosis, for example. The body fluid sample need not be limited to serum, but may also comprise, blood, plasma, cerebrospinal fluid, urine, tears, and saliva, for example.

Elevated free metal levels such as copper and iron can be treated with pharmacologic agents that can either complex such metals, such as thiomolybates and thiotungstenates in the case of copper, and Exjade in the case of iron, or agents that chelate metals such as penicillamine, trientine, clioquinol, EDTA and desferroxamine, for example, or by agents that block absorption of metals, such as zinc acetate.

In a preferred embodiment of the invention, we can greatly improve the deposition selectivity of the device by putting an exclusion barrier film on the working electrode surface. This allows for more rapid and cleaner detection. The specific utility is a size exclusion film for this purpose and a particularly preferred material for use as the barrier film is nafion.

We also have discovered that by using very high concentrations of cysteine in our detection buffer that the copper peak is much much sharper which increases the lower limit of detection. While not wishing to be bound by theory we believe that this is for two reasons: First, copper is exchanged from many low binding species into one copper species which is cysteinyl copper. This then gives a very sharp peak rather than a broad peak from innumerous other copper species. Second, we believe that there is a self-assembly of cysteine on the mercury/carbon working electrode. This provides for enhancement of copper disposition.

### BRIEF DESCRIPTION OF THE DRAWINGS

The invention will be further described with reference to the accompanying drawings, wherein like numerals depict like parts, and wherein:

FIG. 1 is a representation of a potentiostat used to measure free copper in accordance with the present invention;

FIG. 2 is a schematic showing the relationship of the potentiostat of FIG. 1 to the electrode used to measure free copper; and

FIGS. 3A-3C and FIGS. 4-7 are a series of plots including square wave voltammetry (SWV) curves and concentration response curves of copper measurements made in accordance with the instant invention.

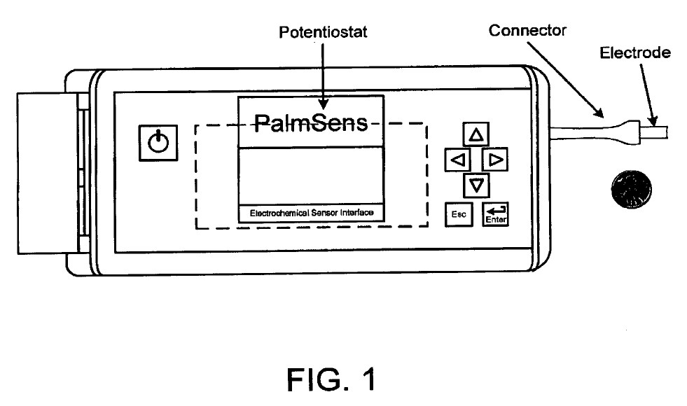

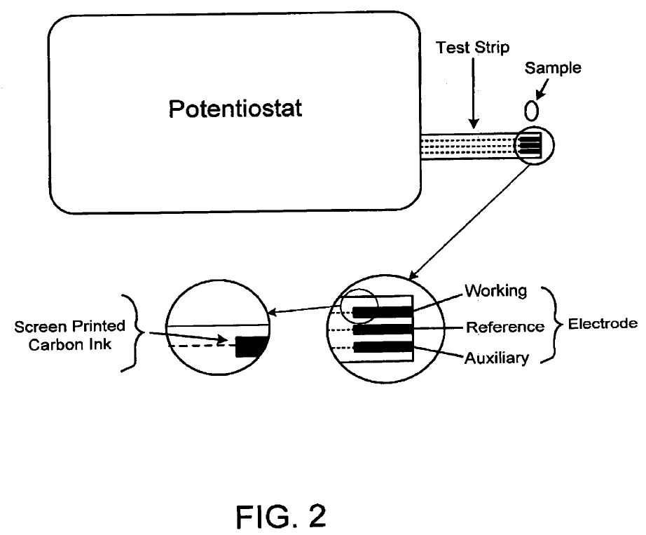

### EXPERIMENTAL

Squitti and researchers have demonstrated that “Free Copper” tracks with the MMSE in Alzheimer's. Historically Free Copper has been measured using methods that are less than direct. We have developed a potentiometric method for directly measuring Free Copper concentrations in serum.

### Hypothesis:

If the time course of copper deposition of copper sulfate on a carbon working electrode can be determined, then this time course should pattern the deposition of Free Copper in serum.

### Method:

1. The following Square Wave Parameters (SWV method) were used:

| | |
|---|---|
| E begin: −0.6 V | |
| E end: −0.0 V | |
| E step potential: 0.003 V | |
| E amplitude: 0.028 V | |
| Freq: 15 Hz | |
| E cond: −0.200 V | t cond: 60 s |
| E dep: −2 V | t dep: 0 to 1600 s |
| E eq: −0.150 | t eq: 30 s |

2. A PalmSens potentiostat available from Palm Instruments, BV, Houten, Netherlands, was used.

3. University of Florence heavy metal electrodes were used. These consisted of carbon, carbon and silver for the working, counter and reference electrodes respectively.

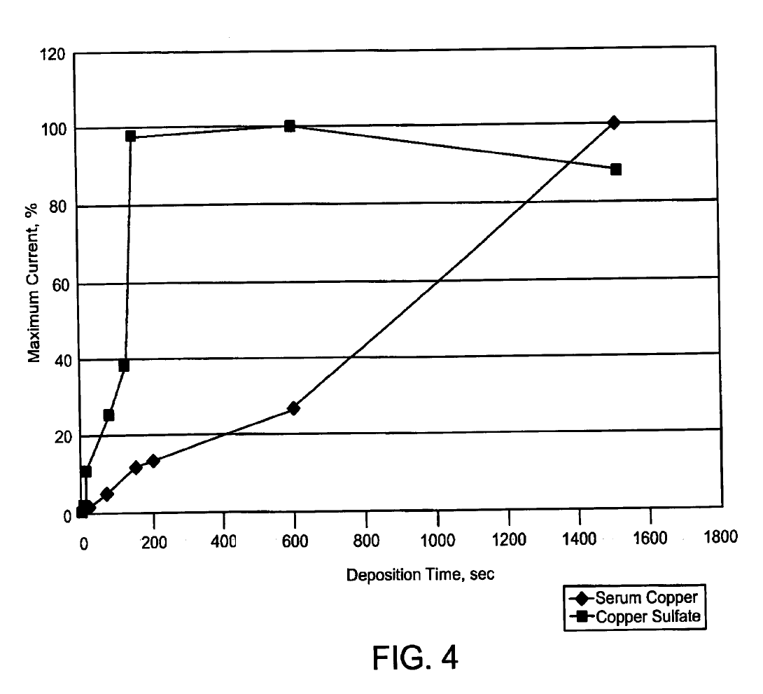

4. For FIG. 4, copper sulfate was measured as a 100 PPB solution in 0.1N HCl.

5. Pathogen-free human serum was purchased from Sigma-Aldrich, Co., St. Louis, Mo.

6. Solutions containing 100 ul of plasma and 900 ul of 0.1N HCl were prepared fresh just prior to its application to the electrode.

7. Following every square wave voltammetry (SWV) measurement, electrodes were cleaned by applying a 100 ul solution of 0.1 N HCl to the electrode using the SWV method in which a deposition potential of 2 V for 60 s was used.

8. The SWV method was used after the application of either 100 ul of copper sulfate or 100 ul of serum solution.

9. A deposition potential of −2V was applied for either 3 or 30 min.

10. A copper SWV signal was observed at about −0.3 volts.

11. The peak height for each copper curve was measured using PalmSens software.

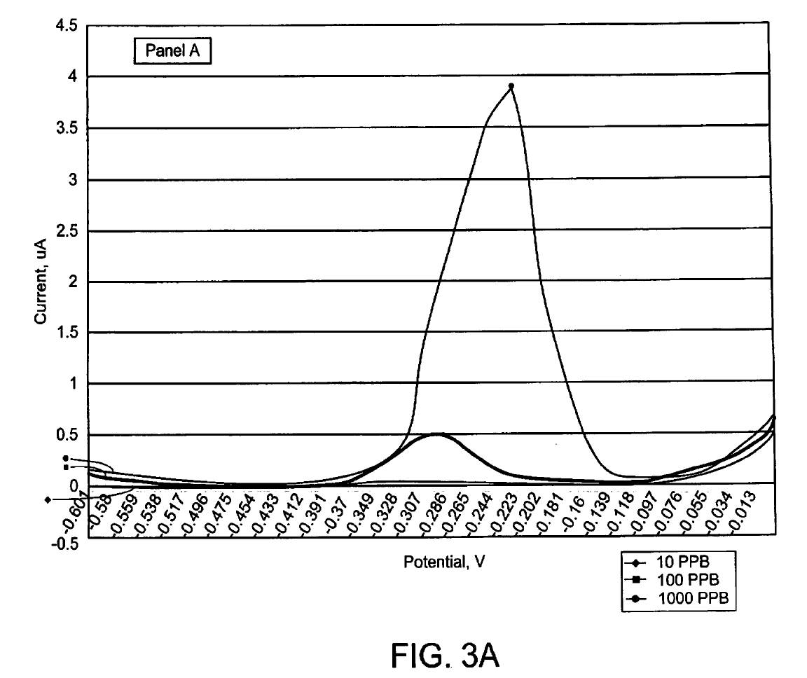

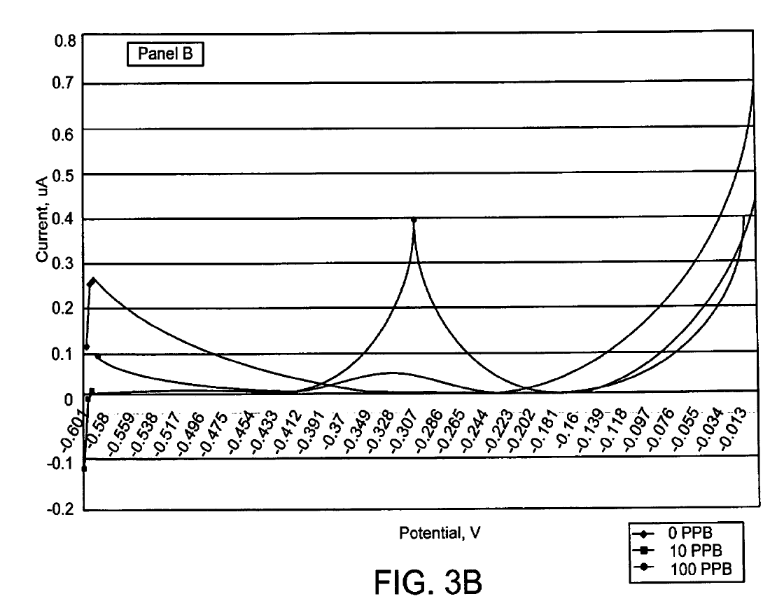

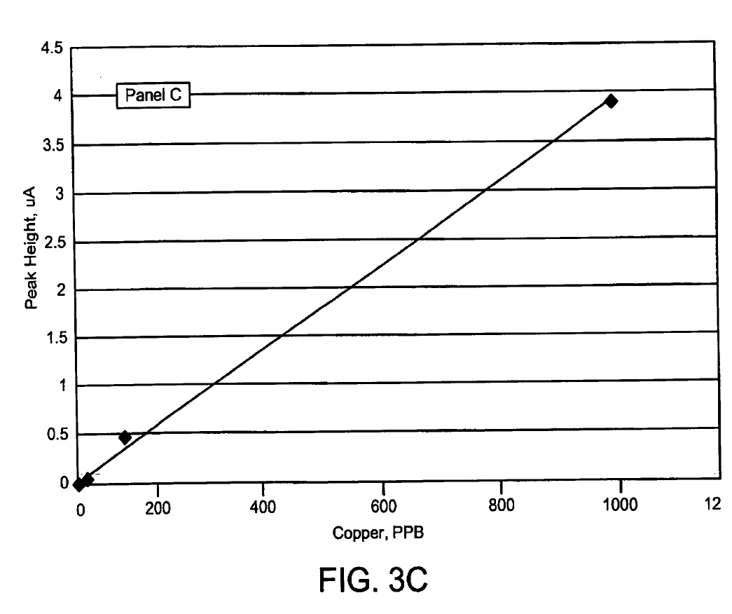

Results are shown in FIGS. 3A-3C, and FIGS. 4-7 as follows:

FIGS. 3A-3C show SWV curves for copper at 0, 10, 100 and 1000 PPB. Within this range, the data gives a linear concentration response range.

FIG. 3A shows square wave voltammetry curves of copper sulfate at 10 PPB (diamonds), 100 PPB (squares) and 1000 PPB (triangles). FIG. 3B shows square wave voltammetry curves of copper sulfate at 0 PPB (diamonds), 10 PPB (squares) and 100 PPB (triangles). And, FIG. 3C shows concentration response curve of data shown in FIGS. 3A and 3B.

### CONCLUSION

Detection of copper as copper sulfate peaked at 3 min. Detection of copper in serum peaked at 30 min. The amount of copper detected in serum at 3 min was only about 13% of the copper detected at 30 min. If we assume that total serum copper has been determined by 30 min, then the free copper (defined by data at 3 min) represents about 13% of the total copper. This value seems reasonable based on other published reports of free copper in serum (20% plus or minus).

FIG. 4 shows SWV Measurement of Copper. In FIG. 4, SWV copper peak heights (as a % of the largest peak height within each time course) were plotted in terms of deposition time (sec).

The diamonds represent copper measured from serum (1 to 10 dilution in 0.1N HCl). The squares represent copper measured as copper sulfate (100 PPB in 0.1 N HCl). The maximum peak current height for 100 PPB copper sulfate was about 1.3 uA. The maximum peak current height for copper serum was about 1 uA. Conclusion: Copper sulfate shows a monophasic response. Serum copper shows a multiphasic response. The free copper response is over by 200 sec. For serum copper, peak height saturation is achieved by 30 minutes.

The maximum peak current height for 100 ppb copper sulfate was about 1.3 uA. The maximum peak current height for copper serum was about 1 uA. Conclusion: Copper sulfate shows a monophasic response. Serum copper shows a multiphasic response. The free copper response is over by 200 sec. For serum copper, the free copper phase represents 13% of total copper if saturation is reached by 1600 seconds. This is reasonable for free copper (non-ceruloplasmin copper) reported in the literature.

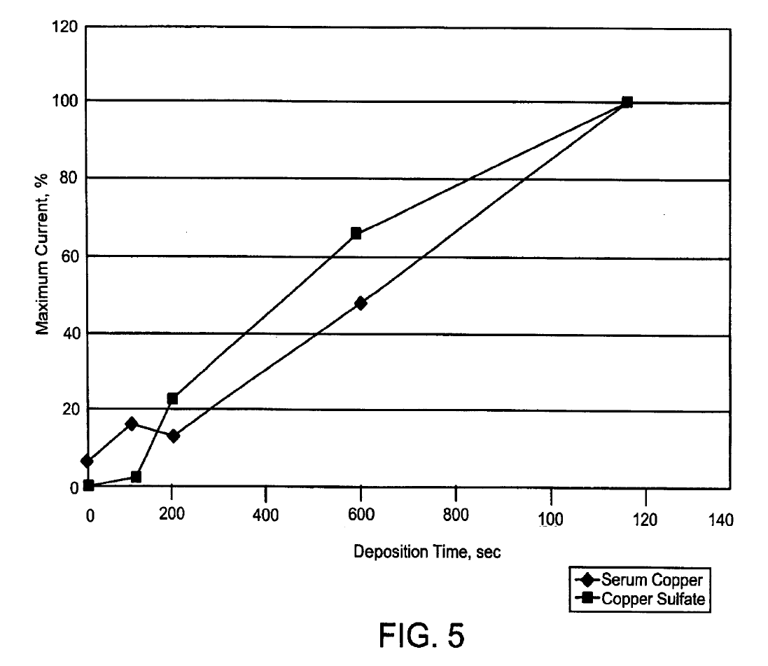

FIG. 5 shows SWV measurement of copper. Diamonds represent copper measured from serum (1 to 10 dilution in 0.1N HCl). Squares represent copper measured as copper sulfate (100 PPB in 0.1 N HCl). Conclusion: The initial rate for measurement of copper sulfate is superimposible with the initial rate for measurement of serum copper.

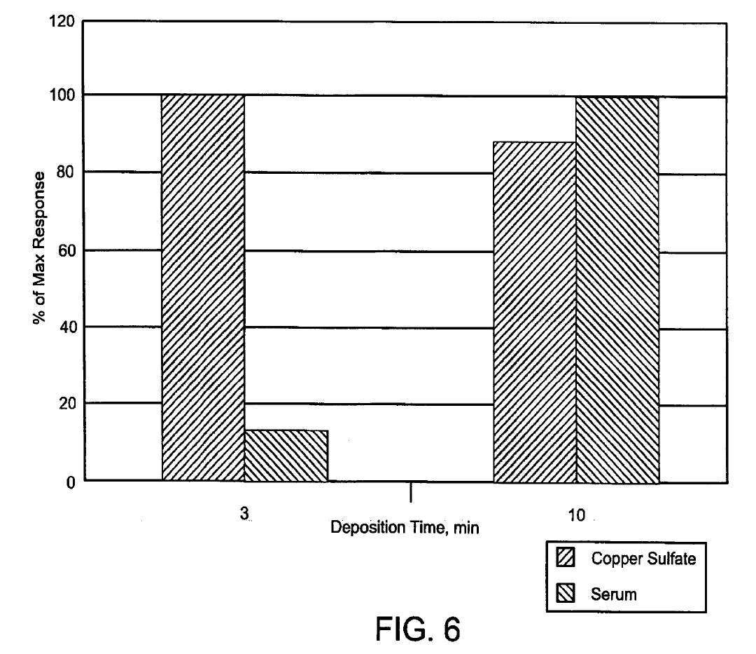

FIG. 6 shows electrochemical detection of copper as copper sulfate (blue) and in serum (red) after 3 and 30 min of deposition.

Light bars represent copper measured as copper sulfate (100 PPB in 0.1 N HCl). Dark bars represent copper measured from serum (1 to 10 dilution in 0.1N HCl). The maximum peak current height for 100 PPB copper sulfate was about 1.3 uA. The maximum peak current height for copper serum was about 1 uA.

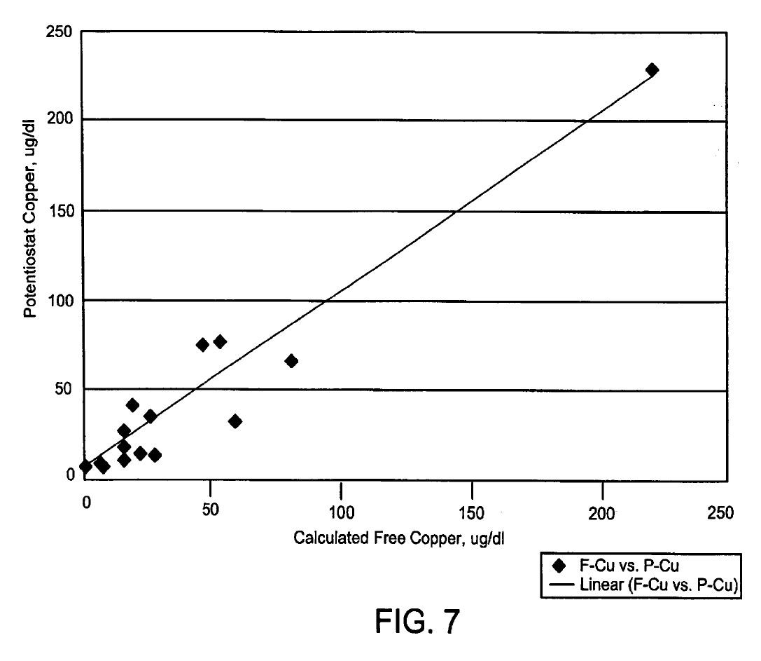

FIG. 7 shows analysis of free copper in serum from Wilson's Patients. Serum samples from Wilson's Patients were provided. Free copper was determined in two ways. One way was based on a calculation measuring total copper by atomic absorption and subtracting the bound copper based on a measurement of ceruloplasmin in each sample (X-axis). The other way (Y-axis) was based on the measurement of free copper using a potentiostat by the method described under Experimental. The correlation coefficient describing the linear relationship between both sets of data is r=0.96.

By virtue of the present invention's ability to directly measure free metal levels in bodily fluids, the number of diseases attributed to such elevated free metal levels is not limited to the scope of the diseases listed above. In fact, the present invention permits the use of direct metal measurement as described by the invention herein to serve as the primary diagnosis of disease itself, the disease being an elevated level of free metal, such as copper, iron or zinc, for example. Until the present invention, it has been difficult to diagnose diseases involving elevated free metals pools because total metal levels in serum, for example, may vary from person to person and may rise and fall based upon acute conditions thereby undermining the significance of such measurements. Generally, however, binding metalloproteins, such as ceruloplasmin and transferrin should also rise and fall. However, as has already been described currently available methods of estimated free metal are highly inaccurate and can be easily overlooked due to an impaired metal binding ability of such metalloproteins due to either genetic (as in the case of Wilson's disease), environmental factors, overload, impaired protein foilding, age-related or infectious agents which may alter the optimal conformation and metal binding potential and reductive and protective capacity of such metalloproteins. Accordingly, the present invention may be used to diagnose any disease which may be caused by elevated free metal pools by directly measuring the free metal levels in bodily fluids.

In a preferred embodiment, the present invention can also be used to determine and titrate the appropriate dose and course of treatment of a pharmacologic agent that either complexes, chelates or blocks the absorption of metals of interest. Since certain metals, such as copper and iron, are essential trace metals, it has heretofore been difficult determine the appropriate dose and course of treatment with agents that complex, chelate or block the absorption of such metals for example. (Indeed, in certain instances supplementation with such metals may even be the best treatment course). As a result, chelation therapy as currently practiced by the art is all too often associated with instances of metal deficiency, which can manifest as leucopenia and anemia, for example. Patients undergoing chelation therapy require careful monitoring by their treating physicians and even under the best supervision instances of metal deficiency are still common. Such instances are due to the fact that, until the present invention, treating and prescribing physicians lacked an accurate and reliable (and preferably rapid as in point of care) method to directly measure free and otherwise available metal levels in patients requiring treatment. Instead, they rely on a process of trial and error and careful supervision which results in cases of essential metal deficiency and is time consuming and burdensome on the physicians, patients, laboratories and the healthcare system. Accordingly, it is a feature of the present invention to provide a method of determining and initial, adjusted and/or titrated dosage amount and regimen of an anti-metal agent that is based upon a direct measurement of a metal of interest. Thus, the present invention's direct metal measurement methodology can be incorporated into a package insert and prescribing information of an anti-metal agent to assist treating physicians and others in selecting, adjusting and titrating the appropriate dose, regimen and course of treatment of an anti-metal therapy so as to maintain an appropriate therapeutic range or index. Such methodology may involve calculations based upon other parameters such as body weight, free and total metal levels, as well as extrapolations determined by clinical trials or clinical experience.

1. An analytical apparatus for detecting non-covalently bound metals (free metal) concentrations in bodily fluids.

2. The apparatus of claim 1, wherein the metal is selected from the group consisting of copper, iron, zinc, manganese, cadmium and selenium.

3. The apparatus of claim 1, comprising a two or three electrode square wave driven potentiostat.

4. An apparatus of claim 1, wherein the electrode system comprises a working electrode, a reference electrode, and a counter or auxiliary electrode or a combined reference and counter or auxiliary electrode.

5. The apparatus of claim 4, wherein at least one of the electrodes is formed of materials selected from the group consisting of carbon, gold, silver, silver chloride, platinum and palladium.

6. The apparatus of claim 4, including a potential source for applying a voltage to the working electrode that is more negative than an oxidation potential of copper.

7. The apparatus of claim 4, including a potential source for creating a negative potential between the working electrode and the counter or auxiliary electrode.

8. The apparatus of claim 4, including a potential source for applying deposition potential voltages to the working electrode that vary decreasingly over time below the oxidation potential of copper.

9. The apparatus of claim 4, including a potential source for applying deposition potential voltages to the working electrodes that vary over time.

10. A detection method which comprises discerning pools of non-covalently bound (free metal) in bodily fluids, wherein the fluids are tested for varying kinetic rates of deposition of said metal from agents which bind said metal at different affinities or are tested for kinetic rates of deposition as determined by decreasing deposition potential.

11. The method of claim 10, including the step of controlling kinetic rate of deposition by the incorporation of a size dependent barrier film, preferably nafion on a surface of the working electrode, and/or determining rate of deposition of said metal from a metal binding agent as a function of the metal binding agent.

12. The method of claim 11, wherein the metal comprises copper, and the binding agent comprises albumin, and including the step of determining rate of deposition of copper bound to albumin relative to ceruloplasmin.

13. The method of claim 10, wherein discernment of pools of said metal is based on varying kinetic rates of deposition as determined by increasing the time of deposition for any given potential.

14. The method of claim 10, wherein the metal comprises copper, and comprising one or both of the following features:
(a) discernment include those in which the deposition of copper from a copper binding agent is achieved more readily due to the binding constant between copper and a selected copper binding agent being lower than that of another copper binding agent, and
(b) deposition rates of weakly bound copper species are normalized by exchange with a chelator in large quantity that produces a primarily single copper species with high rate deposition characteristics such as cystein.

15. The method of claim 10, wherein the metal is selected from the group consisting of copper, iron, zinc, manganese, cadmium and selenium.

16. An electrochemical method for detection of copper in which copper is deposited on an electrode which comprises detecting copper after deposition of the copper has occurred, wherein the standardization is based on the introduction of an internal standard into the assay system, and including the step of introduction of an internal standard that does not interfere with the detection of copper.

17. The method of claim 16, including the step of introduction of an internal standard that reflects the amount of copper deposited on an electrode as the amount of copper varies within a fluid sample over a large range.

18. The method of claim 16, wherein the internal is selected from the group consisting of another transition metal, organic oxidative and reductive molecules, and combinations thereof.

19. The method of claim 16, wherein the internal standard includes a material that normally is not found in the fluid being tested.

20. The method of claim 16, wherein the internal standard comprises cadmium.

21. The method of claim 16, wherein the internal standard is a material that reflects the amount of copper in a sample external to the electrode.

22. The method of claim 16, wherein the internal standard is an integral part of an electrode.

23. A method of diagnosing a disease characterized by an elevated labile metal pool in an individual or animal or by an elevated nontransferrin bound iron pool in an individual or animal.

24. The method of claim 23 in which the metal is selected from the group of copper, iron, zinc, manganese, cadmium and selenium.

25. A method of determining the appropriate dose, regimen and course of treatment of a copper lowering agent which comprises directly measuring free copper in a fluid sample from that individual.
